(19) United States
(12) Patent Application Publication
(10) Pub. No.: US 2025/0199012 A1
(43) Pub. Date: Jun. 19, 2025

(54) SYNERGISTIC CONTROL AND DYNAMIC ASSEMBLY OF VISCOELASTIC NETWORKS AND BIOMOLECULAR CONDENSATES BY AQUEOUS LIQUID-LIQUID PHASE SEPARATION AND LIQUID-SOLID PHASE SEPARATION (AQLL-LS PS2)

(71) Applicant: The University of Hong Kong, Hong Kong (CN)

(72) Inventors: Huanqing CUI, Hong Kong (CN); Ho Cheung SHUM, Hong Kong (CN); Yage ZHANG, Hong Kong (CN); Shipei ZHU, Hong Kong (CN); Jingxuan TIAN, Hong Kong (CN)

(21) Appl. No.: 18/844,463

(22) PCT Filed: Feb. 22, 2023
(86) PCT No.: PCT/CN2023/077616

§ 371 Date: Sep. 6, 2024

Related U.S. Application Data
(60) Provisional application No. 63/269,422, filed on Mar. 16, 2022.

(51) Int. Cl. G01N 33/68; C08L 5/02; C08L 33/24; C08L 89/00; G01N 33/53

(52) U.S. Cl. CPC G01N 33/6875; C08L 5/02; C08L 33/24; C08L 89/00; G01N 33/5308; C08L 2201/06; C08L 2207/02; C08L 2207/53

(57) ABSTRACT

A biological network mimic for investigating subcellular structures and their interaction with biomolecular condensates is presented. The mimic is a stimulus-responsive polymer and a non-responsive polymer in an aqueous two-phase system (ATPS). One effective mimic is an aqueous two-phase system (ATPS) that combines poly (N-isopropylacrylamide) (PNIPAM) and dextran (DEX). The ATPS mimic, displays ultrasensitive thermo-induced aqueous liquid-liquid phase separation and liquid-solid phase separation (AqLL-LS PS²). Diverse structures, including networks, hollow spheres, and spinodal decomposition-like patterns, are generated by regulating component concentrations and temperatures. These structures are thermally reconfigurable. Networks can melt fused in sarcoma (FUS) condensates. The mimics provides methods to examine potential treatments of neurode-generative diseases by dissolving pathologically relevant biomolecular condensates.

### BACKGROUND OF THE INVENTION

Living cells have diverse subcellular hierarchical structures, such as liquid organelles and cytoskeletal networks, that can dynamically assemble, dissociate, and reorganize in response to different physiological signals. These subcellular structures regulate essential cellular activities, including protein synthesis, RNA metabolism, signal transduction, ribosome biogenesis, asymmetric cell division, and cell movements. Subcellular structures can interact mutually; for instance, the cytoskeleton can regulate the intracellular transport of vesicles and adjust the size distribution of membraneless organelles. Subcellular hierarchical structures are precisely modulated through a delicate balance of dynamic assembly to control their functions. Undesired and unregulated assemblies of biomolecules into densely packed supramolecular structures are often associated with diseases. Understanding dynamic synergistic interactions among these subcellular structures is vital. Despite extensive efforts over the past few decades, characterize parameters that dominate the dynamic assembly and dissociation of subcellular hierarchical structures in vivo remains challenging, as does reproducing similar dynamics and interactions.

Synthetic models have been constructed to understand and quantitatively visualize these dynamic structures and their interactions. These models include aqueous droplets dispersed within another continuous aqueous phase, surfactant-stabilized all-aqueous double emulsions in oil, giant lipid vesicle-stabilized phase-separated aqueous droplets in another aqueous phase, and lipid vesicle-stabilized all-aqueous emulsion droplets. For a truly representative system, oil phases would be excluded, as the oil inhibit the activities of diverse biomolecules, such as enzymes. Aqueous two-phase systems (ATPSs) have emerged as promising in vitro models owing to their all-water and biocompatible environment. ATPSs form via phase separation of an aqueous solution containing two incompatible components, such as two polymers, one polymer and one salt, or two salts, above a critical concentration or temperature. The partitioning and compartmentalization of biomolecules renders ATPSs as ideal systems for investigating biochemical reactions. Although ATPSs have been extensively used to mimic the structure and function of living cells, studies have focused primarily on mimicking liquid-like organelles. Other subcellular structures that coexist with organelles, such as biological networks, as well as their functions, are still in their infancy.

A biological network, such as cytoskeleton, which is dynamic and highly complex, can rapidly assemble, dissociate, and remold to maintain cell morphologies and transport biomolecular condensates. An understanding of the working principle of dynamic network structures in vitro provides insights into how the biological network takes part in the essential biological processes and precisely triggers the formation of dynamic networks on-demand. To construct networks in vitro, a variety of strategies have been devised, such as addition of pore-forming agents, phase inversion, and emulsion templating. Currently, networks generated using existing approaches are static, unlike their dynamic counterparts in cells. Therefore, building biocompatible and controlled networks to study their dynamic interactions with biomolecular condensates is crucial. Hence, an ATPS that mimics the dynamic assembly and dissociation of the cytoskeleton and their synergistically interaction with biomolecular condensates is desirable.

### BRIEF SUMMARY OF THE INVENTION

An embodiment is directed to a biological network mimic that is an aqueous two-phase system (ATPS) that includes two immiscible polymers, at least one of these polymers is a stimulus-responsive polymer. Multiple structures including solid spheres, porous networks, hollow spheres, or core-shell spheres are phase separated under external stimulation. The stimulus can be a temperature change, a pH change, irradiation with light, or the application of an electric or magnetic field. The biological network mimic can include a photothermal agent. The photothermal agent can be gold nanorods (GNRs), graphene, MXene, and/or carbon nanotubes. In an exemplary embodiment, the stimulus-responsive polymer is poly(N-isopropylacrylamide) (PNIPAM), and the non-responsive polymer is dextran (DEX). PNIPAM chains reconfigure between hydrophilic coils and hydrophobic globules under the thermal stimulus. Other stimulus-responsive polymers that display similar reversible hydrophilic-to-hydrophobic transition can be: Elastin-like polypeptides or poly(N-vinylcaprolactam) for the stimulus being a temperature change; poly(acrylic acid) or hyaluronic acid for the stimulus being a pH change; or a spiropyran-, azobenzene-, or dithienylethene-derived polymer for the stimulus of an irradiation with light. The phase-separated structures can be solid spheres, porous networks, hollow spheres, or core-shell spheres. The formation of these structures is reversible.

Another embodiment of the invention is directed to a method for probing interactions between dynamic networks and biomolecular condensates in vitro. Using a biological network mimic and at least one biomolecule, the interactions are observed upon stimulating the biological network mimic. In an exemplary example, the biological network mimic is a combination of poly(N-isopropylacrylamide) (PNIPAM) and dextran (DEX) containing a FUS protein, where the structures and compositions within structures undergoes changes with temperature.

### BRIEF DESCRIPTION OF THE DRAWINGS

FIG. 1A shows the synthesis of PNIPAM by radical polymerization.

FIG. 1B shows a GPC trace of PNIPAM obtains from by the radical polymerization of FIG. 1A.

FIG. 1C shows photographs of the thermally reversible phase separation of aqueous PNIPAM (0.5 wt %).

FIG. 1D shows a plot of the light transmittance of a 0.5 wt % PNIPAM solution over the temperature ranging from 25° C. to 37° C.

FIG. 1E shows plot for the binodal curve of the PNIPAM (103 k)/DEX (10 k) system.

FIG. 1F shows a photograph of a PNIPAM/DEX solution at a concentration displaying a single uniform phase.

FIG. 1G shows a microscope image of the PNIPAM/DEX solution of FIG. 1F displaying no phase-separated droplets.

FIG. 1H shows a photograph of a phase separated PNIPAM/DEX mixture at a concentration above the binodal curve showing a sharp aqueous-aqueous interface.

FIG. 1I shows a microscope image displaying phase-separated DEX droplets in the PNIPAM-rich phase.

FIG. 1J shows the binodal curve and tie line of the PNIPAM (103 k)/DEX (10 k) system.

FIG. 2A shows microscope images of the thermo-responsive behaviors of a PNIPAM/DEX solution at non-equilibrium states where a homogeneous single phase of PNIPAM (2.5 wt %)/DEX (2.5 wt %) solution under bright field at 25° C., block (a), transforms into a phase-separated PNIPAM coacervates under bright field (b) rhodamine channel (c), and (d) a merged channel of (b) and (c), at 35° C.

FIG. 2B shows a bar chart for the size distribution of PNIPAM coacervates in the ATPS of FIG. 2A.

FIG. 3 shows microscope images of the thermo-responsive behaviors of a PNIPAM/DEX solution at non-equilibrium states where phase separated DEX droplets in PNIPAM (5 wt %)/DEX (5 wt %) solution under bright field at 25° C. is shown in block (e) with the formation of PNIPAM networks and PNIPAM coacervates under bright field (f), rhodamine channel (g), and (h) merged channel of (f) and (g), at 35° C.

FIG. 4 shows microscope images of the thermo-responsive behaviors of a PNIPAM/DEX solution at non-equilibrium states where phase-separated PNIPAM droplets in PNIPAM (0.5 wt %)/DEX (9.5 wt %) solution under bright field, block (i) at 25° C. with the formation of hollow spheres under bright field (j), rhodamine channel (k), and (1) merged channel of (j) and (k), at 35° C.

FIG. 5 shows a photograph and microscopic images of the thermo-responsive behaviors of PNIPAM (5 wt %)/DEX (5 wt %) solution at equilibrium states, where a PNIPAM/DEX droplet reaches equilibrium in the form of a core/shell structure in hexadecane at 25° C., shown in block (a) with phase-separated PNIPAM droplets in core DEX-rich phase (b), DEX droplets in shell PNIPAM-rich phase (c) and DEX droplets in shell PNIPAM-rich phase under 555 nm excitation (d), whereas at 35° C. block (e), the PNIPAM/DEX droplet in hexadecane de-wets, with core/shell structures in the DEX-rich phase (f), hydrophobic PNIPAM networks in the PNIPAM-rich phase (g) and PNIPAM networks under 555 nm excitation (h), where returning the temperature to 25° C. partially dissolves the PNIPAM networks after 60 s (i), forms spinodal decomposition-like pattern after 90 s (j), and further dissolving the spinodal pattern after 120 s (k), and where a transient phase-separated pattern is fixed by reheating the solution to 35° C. for 30 seconds (1).

FIG. 6 shows schematic drawings and microscopic images illustrating formation of dynamic PNIPAM networks where the schematic diagram of phase-separated DEX droplets in PNIPAM/DEX system at 25° C. block (a) indicates the phase separated DEX droplets from the mixture PNIPAM (5 wt %)/DEX (5 wt %) system under FITC channel (b), RhB channel (c), and merged channel (d) at 25° C., that transforms to schematic diagram (e) of a formed PNIPAM networks in PNIPAM/DEX system at 35° C. corresponding to the generated PNIPAM networks under FITC channel (f), RhB channel (g), and merged channel (h) at 35° C.

FIG. 7 shows microscopic images for a mixture PEG (5 wt %)/DEX (5 wt %) system without hydrophobic interactions does not undergo transformation in respond to temperature changes.

FIG. 8A shows a bar chart of the viscosities of 5 wt % PNIPAM solution and 5 wt % DEX solution, and separated PNIPAM-rich phase, and DEX-rich phase of PNIPAM (5 wt %)/DEX (5 wt %) system.

FIG. 8B shows a plot of the variations of elastic modulus and viscous modulus with the temperature at 1 rad/s for the PNIPAM (5 wt %)/DEX (5 wt %) system.

FIG. 8C is a photograph of a vial containing the mixture PNIPAM (5 wt %)/DEX (5 wt %) system at 25° C.

FIG. 8D is a photograph of a vial containing the mixture PNIPAM (5 wt %)/DEX (5 wt %) system at 35° C.

FIG. 8E shows a plot of the variations of elastic modulus and viscous modulus with the angular frequency for the PNIPAM (5 wt %)/DEX (5 wt %) system.

FIG. 8F shows a plot of the change in elastic modulus and viscous modulus at 10 rad/s upon cycling between 25° C. and 35° C. for five cycles indicating reversibility with temperature.

FIG. 8G shows variations of elastic modulus and viscous modulus as a function of shear strain.

FIG. 8H shows variations of elastic modulus and viscous modulus as a function of shear stress.

FIG. 9 shows microscopic images indicating the interactions between dynamic hydrophobic networks and FUS condensates, illustrating (a) bright field and (b) 488 nm excitation of phase-separated FUS condensates (b) in ultrapure water at 25° C., (c) bright field and (d) GFP fluorescence images of the phase-separated FUS condensates in ultrapure water at 35° C., (e) bright field and (f) fluorescence image (GFP channel) of phase-separated FUS condensates in 5 wt % DEX solution at 25° C., (g) phase-separated FUS condensates in 5 wt % DEX solution at 35° C., (h) fluorescence image (GFP channel) of phase-separated FUS condensates in 5 wt % DEX solution at 35° C., (i) bright field and (j) 488 nm excitation of phase-separated FUS condensates in PNIPAM-rich phase at 25° C., (k) bright field and (1) GFP channel images of a generated PNIPAM networks and disassembly of FUS condensates in PNIPAM-rich phase under at 35° C.

FIG. 10 shows confocal images (Rhodamine channel+GFP channel+bright field) of FUS condensates in the PNIPAM-rich phase at (m) 25° C. and after heating at 35° C. for (n) 40 s, (o) 60 s, and (p) for 290 s.

FIG. 11 shows thermally reversible dewetting/wetting of a PNIPAM (5 wt %)/DEX (5 wt %) droplet in hexadecane, wherein subfigure a is schematic diagram of the thermo-reversible dewetting-wetting transition of a PNIPAM (5 wt %)/DEX (5 wt %) droplet in hexadecane; subfigure b is the PNIPAM/DEX droplet turns into a core/shell structure in hexadecane at 25° C. with phase-separated; subfigure c is PNIPAM droplets in the DEX enriched core; subfigure d is DEX droplets in the PNIPAM enriched shell; subfigure e is the PNIPAM/DEX droplet in hexadecane dewets at 35° C., with subfigure f core/shell structures in the DEX enriched core; and subfigure g is hydrophobic PNIPAM networks in the PNIPAM enriched shell.

FIG. 12 shows the dewetting/wetting of PNIPAM (5 wt %)/DEX (5 wt %) droplet at 25° C. and 35° C., respectively. The droplet is injected into hexadecane, and PNIPAM is labelled with rhodamine B. The fluorescence images of subfigure a PNIPAM (5 wt %)/DEX (5 wt %) droplet, and subfigure b smaller phase-separated DEX droplets in the PNIPAM enriched phase, at 25° C. Subfigure c is dewetting of PNIPAM/DEX droplet, and subfigure d is the formed PNIPAM networks in the PNIPAM enriched phase, at 35° C.

FIG. 13 is the schematic diagram of forming dynamic PNIPAM networks by using phase-separated DEX droplets as templates.

FIG. 14 shows regulation of hydrophobic interactions by external stimuli.

FIG. 15 shows the coalescence of phase-separated DEX droplets in PNIPAM/DEX systems comprising a) 5 wt % PNIPAM and 5 wt % DEX, b) 6 wt % PNIPAM and 4 wt % DEX, c) 7 wt % PNIPAM and 3 wt % DEX, at 25° C.

FIG. 16 shows the generation of a viscoelastic network forbids fusion events in PNIPAM/DEX systems with different component concentrations at 35° C. a) 5 wt % PNIPAM and 5 wt % DEX. b) 6 wt % PNIPAM and 4 wt % DEX. c) 7 wt % PNIPAM and 3 wt % DEX.

FIG. 17 shows fluorescence recovery after photobleaching (FRAP) experiments of PNIPAM (5 wt %)/DEX (5 wt %) system at 25° C. and 35° C. a) Representative images showing the phase-separated DEX droplet and the viscoelastic network during FRAP analysis. b) Normalized FRAP profile and c) fluorescence recovery of the phase-separated DEX droplet and the viscoelastic network; n>3, two-tailed t-test applied for statistical analysis.

FIG. 18 shows box plots illustrating the size distribution of FUS condensates in ultrapure water, DEX, and top phase enriched in PNIPAM.

FIG. 19A shows representative fluorescence microscope images of the FUS condensate during the dynamic dissolution process of a FUS condensate triggered by the formation of hydrophobic PNIPAM networks.

FIG. 19B shows the curve diagram illustrating the variation of the FUS condensate size as a function of time during the dynamic dissolution process of a FUS condensate triggered by the formation of hydrophobic PNIPAM networks.

FIG. 20 shows phase-separated FUS condensates in ultrapure water. a) Bright field and b) 488 nm excitation of phase-separated FUS condensates (1.5 μM) in ultrapure water; and c) bright field and d) GFP fluorescence images of the phase-separated FUS condensates (0.75 μM) in ultrapure water.

FIG. 21 shows liquid-liquid phase separation of FUS protein in the top phase enriched in PNIPAM from PNIPAM (5 wt %)/DEX (5 wt %) at 25° C. under a) bright field and b) GFP channel. The generation of PNIPAM networks and disassembly of FUS condensates under c) bright field and d) GFP channel at 35° C. Condensation of 3 μM FUS protein in the PNIPAM-enriched phase from PNIPAM (5 wt %)/DEX (5 wt %) at 25° C. under e) bright field and f) GFP channel at 25° C. The formation of PNIPAM networks and dissolution of FUS condensates under g) bright field and h) GFP channel at 35° C.

FIG. 22 shows liquid-liquid phase separation of FUS protein in the top phase enriched in PNIPAM from PNIPAM (4 wt %)/DEX (6 wt %) at 25° C. under a) bright field and b) GFP channel. The generation of PNIPAM networks and disassembly of FUS condensates under c) bright field and d) GFP channel at 35° C. Condensation of 1.5 μM FUS protein in the PNIPAM-enriched phase from PNIPAM (9 wt %)/DEX (1 wt %) at 25° C. under e) bright field and f) GFP channel at 25° C. The formation of PNIPAM networks and dissolution of FUS condensates under g) bright field and h) GFP channel at 35° C.

FIG. 23 shows the LLPS of FUS protein in the PNIPAM-enriched phase containing different salt concentrations. a) Bright field and b) 488 nm excitation of phase-separated FUS condensates in the top phase enriched in PNIPAM containing 25 mM KCl at 25° C. The generation of PNIPAM networks and disassembly of FUS condensates in the top phase enriched in PNIPAM under c) bright field and d) GFP channel at 35° C. Partitioning of FUS protein into DEX droplets generated from the PNIPAM-enriched phase containing 200 mM KCl without condensation under e) bright field and f) GFP channel at 25° C. The formation of PNIPAM networks and the adhesive of FUS protein on the networks under g) bright field and h) GFP channel at 35° C.

FIG. 24 shows a) Phase-separated FUS condensates at 3 M KCl solution and b) salting out of PNIPAM (5 wt %)/DEX (5 wt %) system at 3 M KCl solution.

### DETAILED DISCLOSURE OF THE INVENTION

In an embodiment, a biological network mimic is a mixture of a stimulus-responsive polymer and a non-responsive polymer in an aqueous two-phase system (ATPS) as a cytoskeleton dynamic assembly and dissociation mimic. In an embodiment the polymers can be poly(N-isopropylacrylamide) (PNIPAM) and dextran (DEX), which promotes synergistic interactions with biomolecular condensates. PNIPAM is a stimuli-responsive polymer able to reversibly change configuration from hydrophilic coils to hydrophobic globules in response to temperature. The PNIPAM/DEX system displays ultrasensitive phase separation, with a transition temperature around 31° C., that is similar to the lower critical solution temperature (LCST) of PNIPAM and close to the human body temperature. This system provides a method, according to an embodiment, for investigating the interactions between dynamic networks and biomolecular condensates in vitro. Upon setting the temperature to 35° C., adjusting component concentrations, the PNIPAM/DEX system forms diverse structures, including solid spheres, porous networks, hollow spheres, and core-shell spheres. These ATPS-templated structures, such as the porous networks, are reconfigurable due to the thermally reversible transitions of PNIPAM chains. By decreasing the temperature to 25° C., the hydrophobic networks dissolve gradually, forming transient patterns similar to those observed in spinodal decomposition. The transient phase-separated patterns can reform by heating the ATPS to 35° C. The ATPS-templated porous networks are generated through aqueous liquid-liquid phase separation and liquid-solid phase separation (AqLL-LS PS²).

Triggered hydrophobic interactions induce clustering of nonpolar molecules and minimize contact with surrounding aqueous environments forms porous networks via a liquid-to-solid transition with aqueous droplets providing spacers. The generated networks can interact with the biomolecular condensates, such as fused in sarcoma (FUS) protein. FUS is an essential RNA-binding protein associated with amyotrophic lateral sclerosis (ALS) that participates in RNA processing and transcription. Mixed with the PNIPAM/DEX system at 25° C., FUS partitions into phase-separated DEX droplets and form micron-sized condensates. The liquid-liquid phase separation of FUS proteins is driven by the reinforced multivalent interactions, especially the hydrophobic interactions, between intrinsically disordered regions due to the dilution of salt, which reduces electrostatic screening. Stable hydrophobic PNIPAM networks are formed when the temperature reaches 35° C. Phase-separated FUS condensates melt within five minutes after physically contacting the generated hydrophobic networks due to the viscoelastic constraints and hydrophobic interactions. These ATPS forms long-live hydrophobic networks via AqLL-LS PS² and efficiently melts phase-separated FUS condensates. The ATPS dynamic networks, according to an embodiment, facilitate the investigations of biomolecular condensation in free and constrained environments, and precisely regulate biomolecular condensates on-demand. The ATPS shows aqueous phase separation phenomena with richness and complexity and the ability to dissolve pathologically relevant protein condensates, in a manner useful for the discernment of routes for suppression of pathological precipitates.

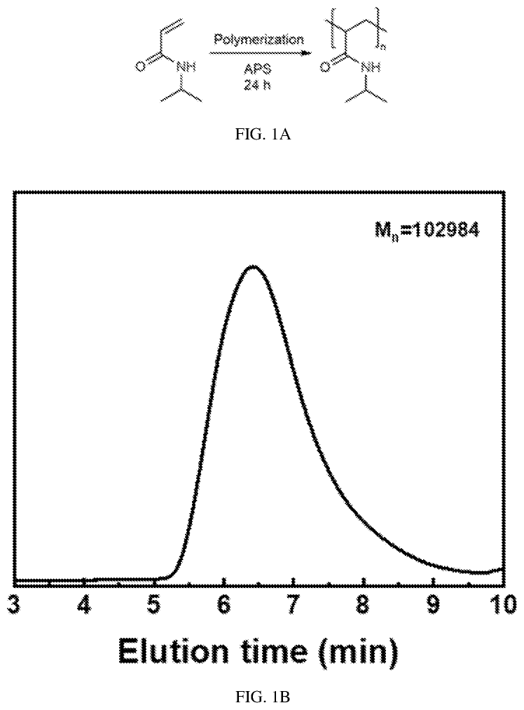

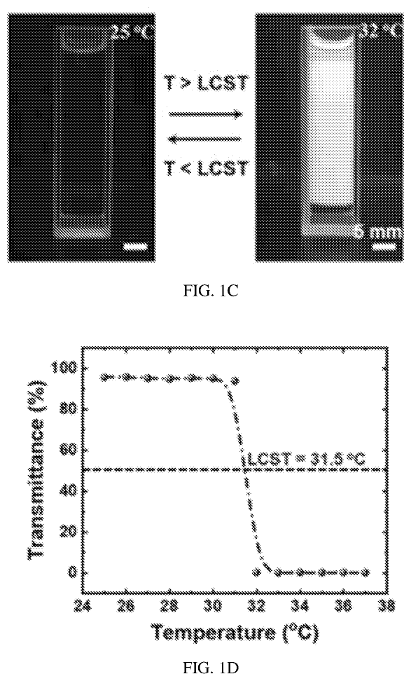

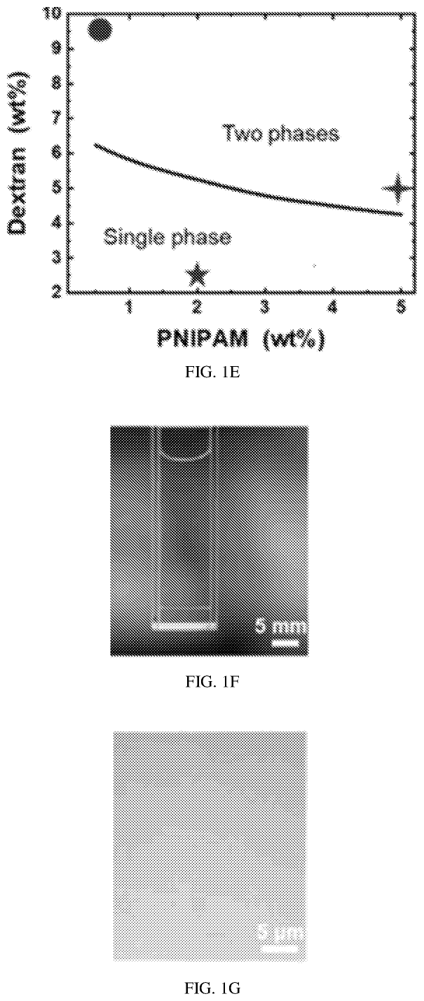

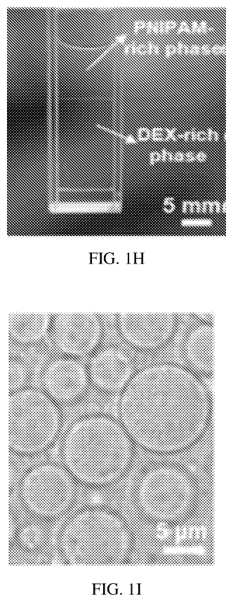

Thermo-responsive PNIPAM, selected as a component of the ATPS according to an embodiment, is synthesized by radical polymerization as shown in FIG. 1A, where the GPC, as shown in FIG. 1B is consistent with a number-average molecular weight of around 103 k. The PNIPAM forms an aqueous solution that is transparent at 25° C. and turns milky-white when the temperature is increased to 32° C., as indicated in FIG. 1C. The light transmittance of a 0.5 wt % PNIPAM aqueous solution was recorded at various temperatures to quantify the transition temperature. As shown in FIG. 1D, when the temperature is below 32° C., the transmittance is higher than 95%, but by raising the temperature above 32° C., the transmittance decreases sharply to 0.2%, which corresponds to a reconfiguration of PNIPAM chains from hydrophilic coils to hydrophobic globules results in this transition. The LCST of PNIPAM, defined as the temperature with 50% light transmittance, occurs at 31.5° C., which is consistent with literature values. The PNIPAM (103 k)/DEX (10 k) combination at a concentration below the binodal curve, as shown in FIGS. 1E-1G and 1J, is of a single uniform phase with no phase-separated droplets upon magnification. At concentrations above the binodal curve, the enthalpic penalty overcomes the entropic contribution driving phase separation. After reaching equilibrium, a clear aqueous-aqueous interface is observed, as is shown in FIG. 1H, due to the formation of the new ATPS. Phase-separated DEX droplets are generated in the upper PNIPAM-rich phase, as shown in FIG. 1I. It is worth noting that the binodal curve is unavailable once the temperature is above the LCST of PNIPAM due to the hydrophobicity of PNIPAM chain and insolubility in water.

In other embodiments of the invention other reconfigurable stimuli-responsive polymers systems allow reversible phase separation in response to external stimuli. By selecting stimuli-responsive materials with the specific hydrophilic-to-hydrophobic transition, the hierarchical structures can be precisely triggered by different physio-chemical signals on-demand. For example, adding photothermal agents, such as gold nanorods, graphene, MXene, or carbon nanotubes, into ATPSs containing thermo-responsive materials endow the system with photo-sensitive properties. The desired subcellular structures can be regulated in a contactless and spatiotemporal manner. Other suitable stimuli-responsive polymers that can be employed, alone or included into the ATPS include: Elastin-like polypeptides and poly(N-vinylcaprolactam) to provide other or additional thermo-responsive properties; poly(acrylic acid) with hyaluronic acid for pH-responsive behaviours; and spiropyran-, azobenzene-, and dithienylethene-derived polymers for light responsive systems. Such intelligent ATPS facilitates investigation of interactions between diverse subcellular hierarchical structures and demonstrates their potential to dissolve pathologically relevant biomolecular condensates formed by hydrophobic interactions.

In embodiments, non-limiting thermo-responsive polymers that can be used include, but are not limited to: poly(N-isopropylacrylamide), poly (N-n-propylacrylamide), poly (N-cyclopropylacrylamide), poly (N-isopropyl, N-methylacrylamide), poly (N-ethylacrylamide), poly (N-acryloxy-N-propylpiperazine), poly (N-(L)-(1-hydroxymethyl) propyl methacrylamide), poly(N-2-(methacryloyloxy) ethyl pyrrolidone), poly (N-(3-acryloxypropyl) pyrrolidone), poly (N-(3-methacryloxypropyl) pyrrolidone), poly (N-(2-acryloxypropyl) pyrrolidone), poly (N-(1-methyl-2-acryloyloxy ethyl) pyrrolidone), poly(2-alkyl-2-azoline), poly(2-ethyl-2-oxazoline), poly(2-isopropyl-2-oxazoline), poly(2-n-propyl-2-oxazoline), poly (N, N-dimethylaminoethyl methacrylate), poly (N-vinyl caprolactam), poly (N-acryloyl pyrrolidine), poly (methyl vinyl ether), poly (2-methoxyethyl vinyl ether), poly (2-ethoxyethyl vinyl ether), poly ((2-ethoxy) ethoxy ethyl vinyl ether), poly (propylene oxide), poly (ethylene oxide), poly (oligo (ethylene glycol) monomethyl ether methacrylate), polyphosphazene, Elastin-like polypeptide, and copolymers or derivatives containing the above-mentioned units. In embodiments, non-limiting photothermally responsive materials, which include a composite additive with a photothermal response with the thermo-responsive polymer, can be, but are not limited to: gold nanorods, gold nanoshells, gold nanocages, hollow gold nanospheres, palladium nanosheets, Pd@Ag nanoparticles, Pd@SiO₂ nanoparticles, carbon nanotubes, graphene, reduced graphene oxide, carbon black, black phosphorus, copper sulphide, indocyanine green, Polyaniline, and the above-mentioned materials with chemical modifications. In embodiments, non-limiting magneto-thermally responsive materials, which include a composite additive with a magneto-thermal response with the thermo-responsive polymer, can be, but are not limited to: Fe₃O₄, LaFeCoSi, GdSiGe, LaFe_{11.6}Si_{1.4}C_{0.2}H_{0.7}, La(Fe,Si)₁₃, NiMnGa, MnCoGe_{0.99}In_{0.01}, MnCo_{0.98}Cr_{0.02}Ge, and modified variations thereof. In embodiments, non-limiting electro-thermally responsive materials, which include a composite additive with a electro-thermal response with the thermo-responsive polymer, can be, but are not limited to: graphite, carbon black, carbon nanotubes, carbon fiber, Al-doped ZnO, calcium doped lanthanum chromate, antimony doped tin dioxide, gold, silver, platinum, copper, rhodium, palladium, chromium, indium tin oxide, transparent conductive oxides, polyacetylene, polyaniline, polypyrrole, other metals, and modified variations thereof. In embodiments, non-limiting pH-responsive polymers that can be used include, but are not limited to: poly (acrylic acid), poly (L-glutamic acid), poly (L-histidine), poly (aspartic acid), poly (N, N-dimethylaminoethyl methacrylate), poly (N,N-diethylaminoethyl methacrylate), poly (methacrylic acid), poly (2-ethylacrylic acid), poly (propylacrylic acid), poly (itaconic acid), poly (vinylphosphonic acid), oligo (4-vinyl-phenyl phosphate), poly (styrene sulfonic acid), poly (4-styrene sulfonic acid), poly (4-Vinylbenzeneboronic acid), poly (N-ethylpyrrolidine methacrylate), polyvinyl alcohol, poly (4-vinyl pyridine), polyethylenimine dendrimers, Chitosan, Alginic acid, carboxymethyl cellulose, Hyaluronic acid, or any copolymer or derivative thereof.

Following are examples that illustrate procedures for practicing the invention. These examples should not be construed as limiting.

### Materials and Methods

### Materials

N-isopropylacrylamide (NIPAM), tetramethylethylenediamine (TMEDA), rhodamine B, fluorescein isothiocyanate-dextran (M_{w}=4 kg/mol or 10 kg/mol), N,N-Dimethylformamide (DMF), ultrapure water, polymethyl methacrylate (PMMA, analytical standard for GPC, Mₙ=46,000), polyvinyl alcohol (PVA, M_{w} 9000-10000, 80% hydrolyzed), polyethyleneimine (PEI, Mₙ 5000), polyacrylamide (PAM, Mₙ 40000), ficoll (type 400) and hexadecane were purchased from Sigma Aldrich. Ammonium persulfate (APS) and DEX (M_{w}=10 kg/mol) were bought from Aladdin. FUS proteins labeled with GFP were provided by Prof. Tuomas P. J. Knowles. All reagents were used as received. Deionised (DI) water was used in all the experiments unless otherwise noted.

### Synthesis of PNIPAM

PNIPAM was synthesized by radical polymerization where: 1 g NIPAM was dissolved in 10 ml water and oscillated for 10 mins to form a homogeneous solution; 5 mg APS was added into the solution; the solution sonicated for 2 min for complete dissolution; and 10 uL TMEDA was injected into the pre-polymerization solution to initiate the polymerization. After 24 h, the resulting solution was dialyzed for 72 h and then freeze-dried for 48 h. The resulting PNIPAM power was stored at 4° C.

### Design of ATPS

Different mass fractions of PNIPAM (103 k) and DEX (10 k) were mixed and injected into a chamber for the following observation under a microscope. The chamber was prepared by sandwiching two coverslips with a 120-μm-thick spacer.

The PNIPAM (5 wt %)/DEX (5 wt %) system was prepared by dissolving 0.5 g PNIPAM (103 k) and 0.5 g DEX (10 k) in 9 mL DI water. The resulting solution became homogeneous when oscillated for 10 mins. The homogeneous ATPS (2 μL) was injected into 3 mL hexadecane to avoid evaporation. After standing for 24 h, the droplet reached equilibrium, turned into a core/shell structure and was placed on a transparent hot plate to regulate the temperature. Thermo-responsive behaviors of the equilibrate droplet were observed under a microscope.

The homogeneous PNIPAM (5 wt %)/DEX (5 wt %) system was centrifuged at 8000 rpm for 30 mins or demixed for 24h to obtain the PNIPAM-rich phase and DEX-rich phase. The PNIPAM-rich phase at the top and the dextran-rich phase at the bottom were separated. Finally, the PNIPAM-rich and the dextran-rich phases were injected into two chambers, which were prepared by sandwiching two coverslips with a 120-μm-thick spacer, and the chambers were placed on the transparent hot plate for observation.

### Interactions Between Dynamic Hydrophobic Networks and FUS Condensates

The ALS-associated protein FUS was mixed with different solutions to investigate its LLPS and the interactions with dynamic networks. 1 μL FUS protein (30 μM) was homogeneously mixed with 19 μL bulk solutions, including DI water, 5 wt % DEX, 5 wt % PNIPAM, and the PNIPAM-rich phase of the equilibrated PNIPAM (5 wt %)/DEX (5 wt %) system. The final buffer contains 2.5 mM Tris, 0.05 M KCl, 0.05 mM DTT, and 0.25% glycerol. Each mixture was injected into a chamber, which was prepared by sandwiching two coverslips with a 120-μm-thick spacer, and placed on the transparent hot plate for observation.

### Characterization of Structural and Physical Properties of the PNIPAM/DEX System.

The molecular weight of PNIPAM was measured by using gel permeation chromatography (GPC, Waters 515). DMF was the mobile phase delivered at a rate of 1 mL/min. PMMA (Mₙ=46,000) was used to prepare the calibration curve. The light transmittances of PNIPAM solution (for example, 0.5 wt % PNIPAM solution) at different temperatures were measured by a spectrophotometer (Shimadzu, UV 2600). The binodal curve and the tie-line are obtained by manual dilution of concentrated phase-forming polymers through extensive pipetting followed by waiting for phase separation, then determining the volumes, and weighing the mass of the resulting phases. A camera (EOS 70D) was used to produce digital photos of the prepared PNIPAM/DEX systems. All microscope images and videos were recorded using a fluorescence microscope (Nikon Ti2-E). A confocal laser scanning microscope (Zeiss LSM 700) was applied to take confocal images. FRAP was performed on a Carl Zeiss LSM 880 microscope equipped with a 40× oil immersion objective. The DEX droplet and viscoelastic network labeled with FITC were bleached by a 488 laser at 100% power for 14 s and 46 s, respectively. Post-bleaching images and fluorescence intensity in the bleached region were taken at 2% power of the 488-nm laser every 5 s. All viscosities, sol-gel transition temperatures, elastic and viscous moduli were measured by a rheometer (MCR 302, Anton Paar).

### Thermo-Responsive Behaviors of PNIPAM/DEX System at Non-Equilibrium States

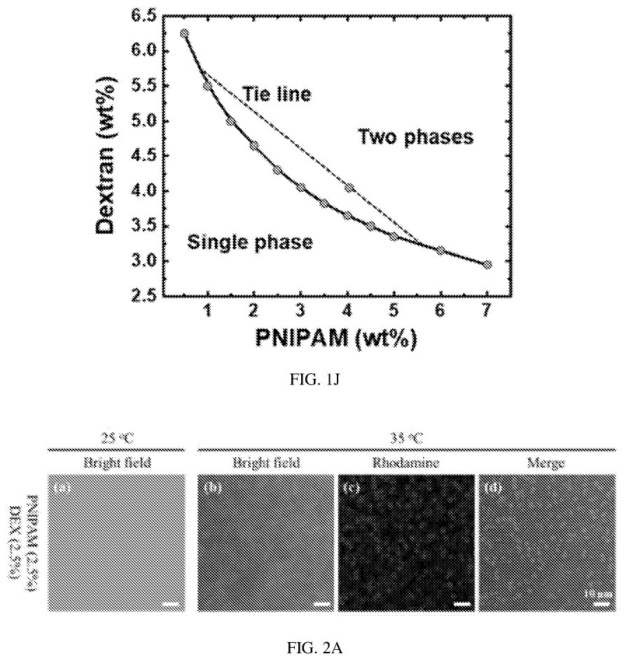

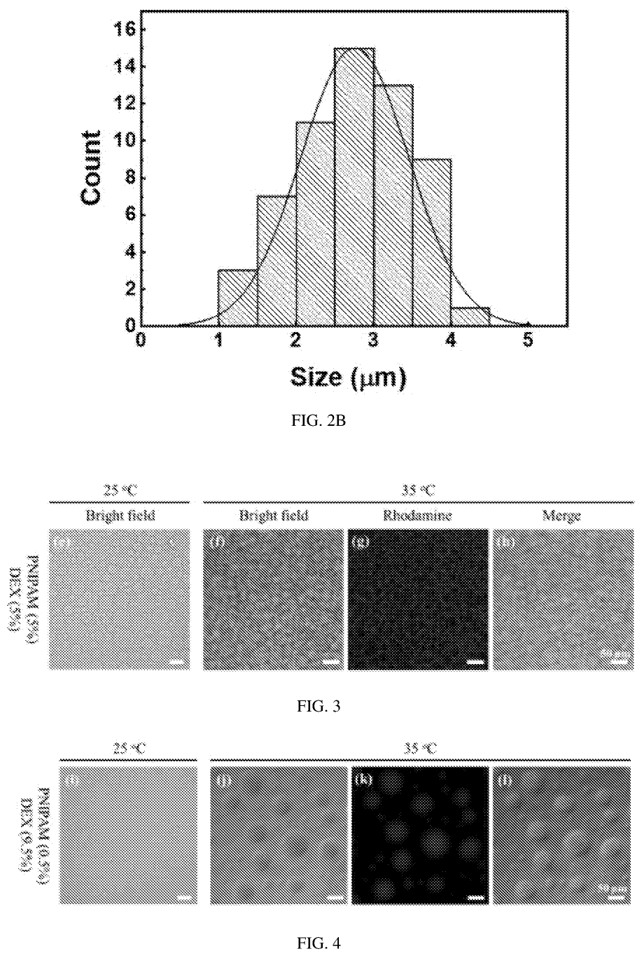

Different compositions of the PNIPAM/DEX system, according to embodiments, show diverse thermo-responsive behaviors at non-equilibrium states, where compositions below the binodal curve, as in FIG. 1E, e.g., 2.5 wt % PNIPAM and 2.5 wt % DEX, display a single homogeneous phase with no phase-separated droplets been observed at 25° C., as shown in channel block (a) of FIG. 2A. When the temperature is increased to 35° C., PNIPAM chains transit from hydrophilic to hydrophobic, and numerous PNIPAM aggregates labeled with rhodamine B are generated, as displayed in FIG. 2A channels (b) through (d). The diameters of the aggregates range from 1.3 to 4.2 μm, as shown in FIG. 2B. As shown in FIG. 3A, for compositions that reside above the binodal curve (e.g., 5 wt % PNIPAM and 5 wt % DEX), DEX droplets phase-separate at room temperature (˜25° C.), as shown in channel (e). By raising the temperature to 35° C., hydrophobic PNIPAM networks and PNIPAM coacervates are formed simultaneously in the continuous and droplet phases, respectively, as shown in FIG. 3 channels (f) through (h). In contrast, a second composition above the binodal curve composed of 9 wt % DEX and 0.5 wt % PNIPAM has different phase separation behaviors at 25° C., the low-concentrated PNIPAM separates from the continuous DEX phase and forms droplets. Inside these PNIPAM droplets, smaller DEX droplets are further separated and coalesced, as demonstrated in FIG. 4 channel (i). By raising the temperature to 35° C., the PNIPAM droplets with smaller DEX droplets inside turns into hollow spheres as shown in FIG. 4 channels (j) through (1). Hence, distinct sophisticated structures can be obtained with this thermal-sensitive ATPS by regulating PNIPAM/DEX concentrations and the system's ambient temperatures.

### Thermo-Responsive Behaviors of PNIPAM/DEX System at Equilibrium States

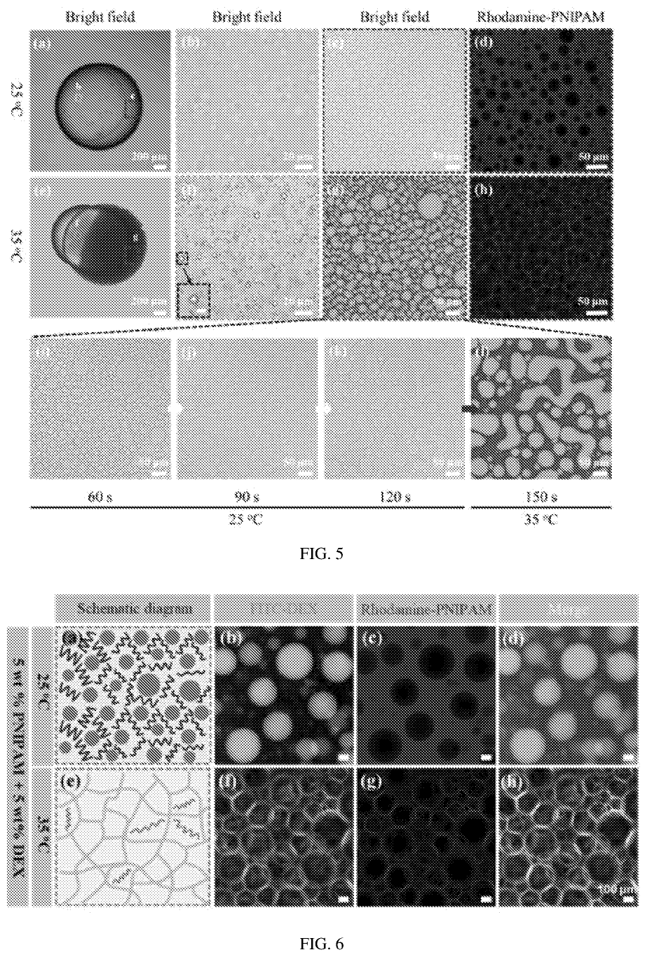

PNIPAM/DEX compositions above the binodal curve of FIG. 1E ultimately reach equilibrium and phase-separate into two composite-rich phases after standing for 24 h. Injecting a 2 μL heterogeneous ATPS (5 wt % PNIPAM and 5 wt % DEX) droplet into 3 mL hexadecane at room temperature is covered by bulk hexadecane, with a smaller density, avoiding any water evaporation from the ATPS. After 24 h, the PNIPAM/DEX droplet reaches equilibrium in the form of a core/shell structure, as shown in FIG. 5 block (a). Smaller PNIPAM and DEX droplets can be observed in the DEX-rich core and PNIPAM-rich shell, respectively, as confirmed by the bright-field images in blocks (b) and (c), as well as the fluorescence images in block (d). The equilibrated ATPS droplet de-wets block (e) with a DEX-rich phase protrudes when the temperature is increased to 35° C. The de-wetting is attributed to the increasing repulsive forces between the hydrophobic PNIPAM and hydrophilic DEX. Core/shell block (f) and network structures blocks (g) and (h) formed in the DEX-rich and the PNIPAM-rich phases, respectively. The de-wetting process is thermally reversible since PNIPAM chains can switch between hydrophilic coils and hydrophobic globules in response to temperature changes. By decreasing the temperature of the same networks to 25° C., the hydrophobic PNIPAM networks dissolve, and the protruding DEX-rich phase retracts. The PNIPAM networks dissolve gradually after cooling at 25° C. for 60 s, block (i) and forms transient phase-separated spinodal decomposition-like patterns after 90 s, blocks (j) and 120 s (k). The generated spinodal pattern can further be fixed by reheating the solution to 35° C., block (1). Such fixation of transient phase-separated spinodal decomposition-like patterns formed from the PNIPAM/DEX system facilitates the investigations of the dynamics of liquid-liquid phase separation, especially the dynamics of the transient spinodal phase separation.

### Formation Mechanism of Dynamic PNIPAM Networks

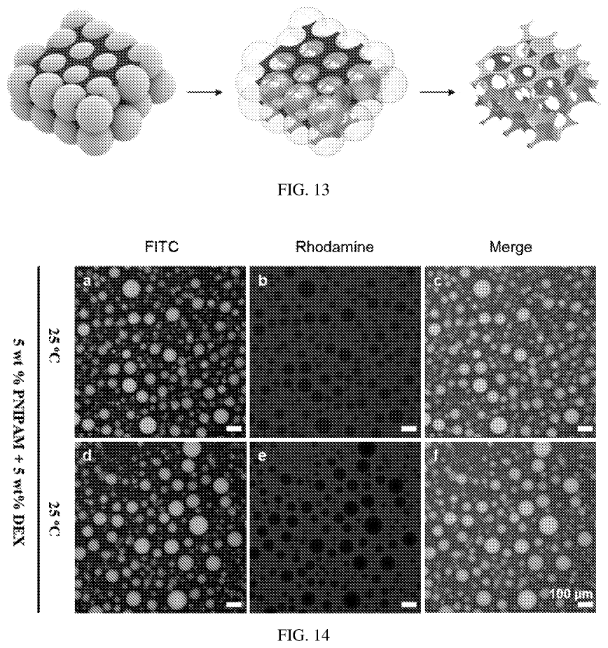

The essential role of dynamic networks in biological processes and precisely regulate their assembly and dissociation on-demand, is reflected in the formation mechanism of the dynamic PNIPAM networks. To work towards regulating the assembly and dissociation of viscoelastic networks on-demand, this application clarifies the formation mechanism of the PNIPAM networks (FIG. 13). For the PNIPAM/DEX system with a concentration above the binodal curve (e.g., 5 wt % PNIPAM and 5 wt % DEX), DEX droplets phase-separate from a continuous phase, as illustrated in FIG. 6 block (a) through (d). Most PNIPAM chains are enriched in the continuous phase, encapsulating phase-separated dextran droplets. When the temperature is higher than the LCST of PNIPAM (31.5° C.), the configuration of PNIPAM chains will transit from hydrophilic coils to hydrophobic globules. To minimize the energy of the system, hydrophobic PNIPAM globules reduce water contact by clustering and forming networks via a liquid-to-solid transition with the aqueous droplets as spacers, blocks (e) through (h). The generated hydrophobic interactions and intramolecular hydrogen bonds between PNIPAM globules strengthen the stability of the PNIPAM networks. The PEG/DEX system as a control system verifies the essential role of hydrophobic interactions, as neither PEG nor DEX displays hydrophobic interactions in response to temperature changes. The system comprising 5 wt % PEG and 5 wt % DEX also displays phase-separated DEX droplets at 25° C., similar to the PNIPAM/DEX system, as shown in FIG. 7 blocks (a) through (c). By increasing the ambient temperature to 35° C., unlike their counterparts in the PNIPAM/DEX system, the phase-separated DEX droplets in the PEG/DEX system remain stable without noticeable shape changes, as shown in Blocks (d) through (f). This confirms that the formation of the dynamic networks in the PNIPAM/DEX system critically depends on the switchable hydrophilic-to-hydrophobic transition of PNIPAM. This hydrophobic interaction is thermally triggered, as the PNIPAM/DEX system only generates networks (FIG. 6) under sufficient temperature changes. The system would remain in its original state, in which DEX droplets are dispersed in PNIPAM continuous phase (as shown in FIG. 14, phase-separated DEX droplets from the PNIPAM (5 wt %)/DEX (5 wt %) system under a) FITC channel, b) RhB channel, and c) merged channel at 25° C.; and coalescence of the phase-separated DEX droplets in the PNIPAM continuous phase under d) FITC channel, e) RhB channel, and f) merged channel at 25° C.), when the temperature remains the same.

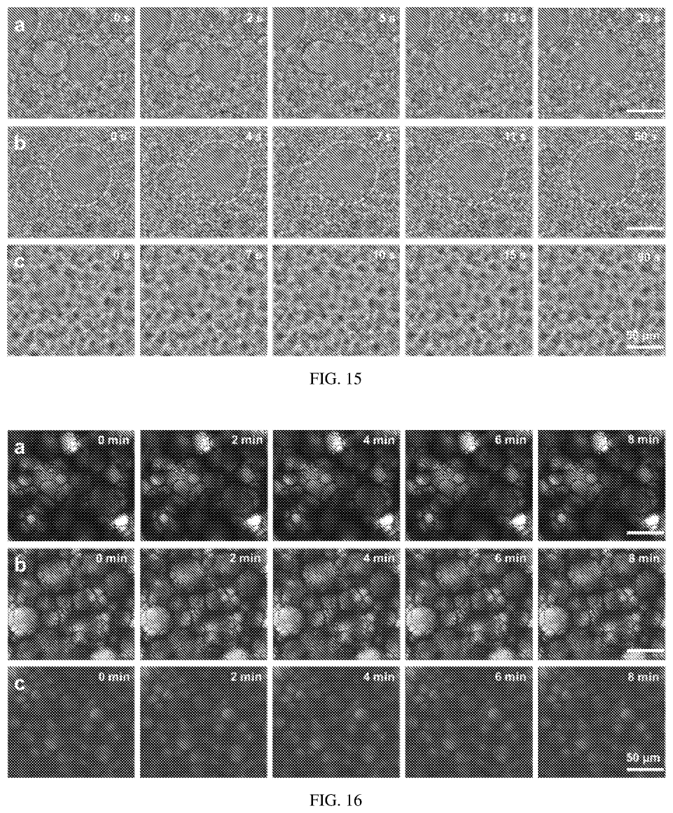

To confirm the viscoelastic networks are generated by AqLL-LS PS², the LLPS dynamics of the PNIPAM/DEX system with different component concentrations are systematically characterized. As shown in FIG. 15, multiple phase-separated DEX droplets are observed in the system containing 5 wt % PNIPAM and 5 wt % DEX at 25° C. The rapid coalescence of phase-separated DEX droplets within 33 s indicates the liquid-like state of the PNIPAM/DEX system. By raising the PNIPAM concentration to 6 wt % and 7 wt %, DEX droplets are still phase-separated and fused at 25° C., with the coalescence time prolonged to 50 s and 90 s, respectively. It may be due to the high viscosity of PNIPAM, which slows down molecular diffusion. When the temperature is increased to 35° C., viscoelastic PNIPAM networks are formed using phase-separated DEX droplets as sacrificial templates (FIG. 16). No fusion events are observed in all three groups within 8 min due to the solid like feature of the formed viscoelastic networks.

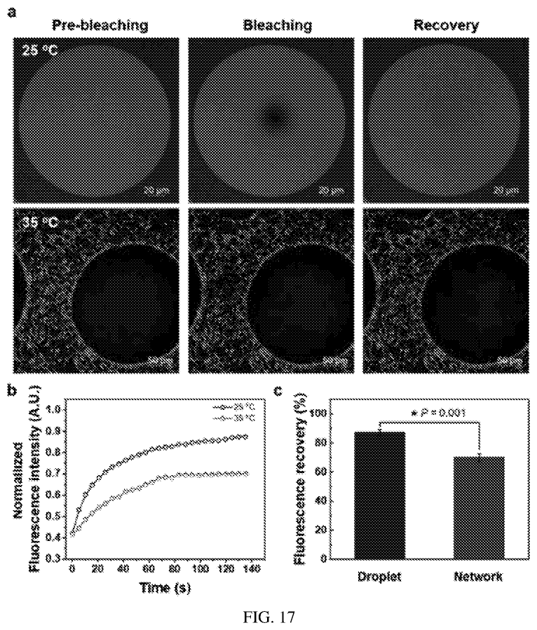

This application further characterizes the dynamics of the phase-separated droplet and viscoelastic network by fluorescence recovery after photobleaching measurements. DEX is labeled with fluorescein isothiocyanate (FITC). For the PNIPAM/DEX system with a concentration above the binodal curve (e.g., 5 wt % PNIPAM and 5 wt % DEX). A phase-separated DEX droplet is observed at 25° C., as shown in subfigure a of FIG. 17. The fluorescence intensity recovers to 87% of the initial intensity within 135 s (subfigure b, c of FIG. 17). The rapid intensity recovery indicates the free diffusion of fluorescent molecules and thus confirms the liquid-like state of the phase-separated DEX droplet. When the temperature is increased to 35° C., a viscoelastic network is formed (subfigure a of FIG. 17). However, only 69% fluorescence is found to be recovered after 135 s (subfigure b, c of FIG. 17) due to the solid-like feature of the formed viscoelastic network.

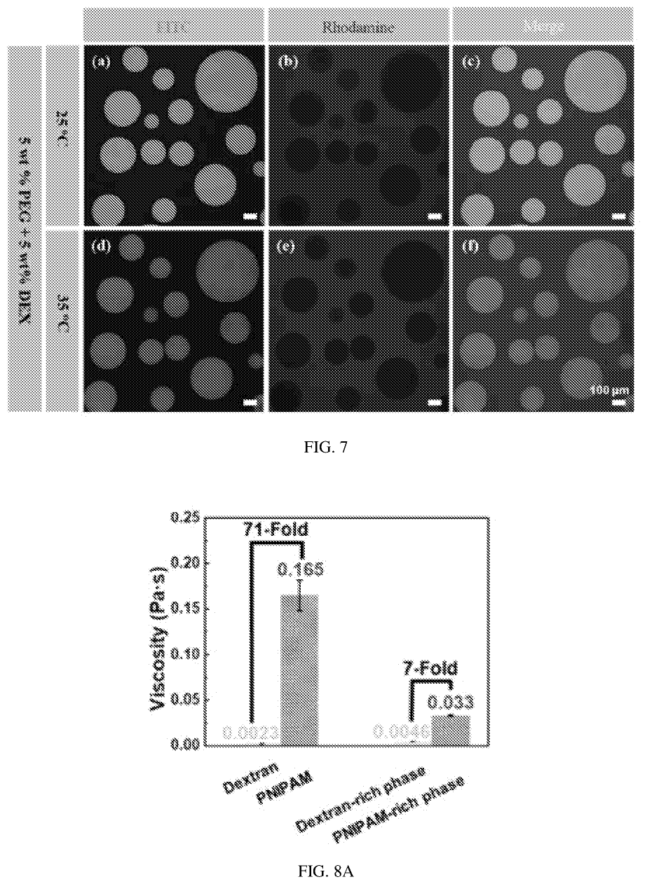

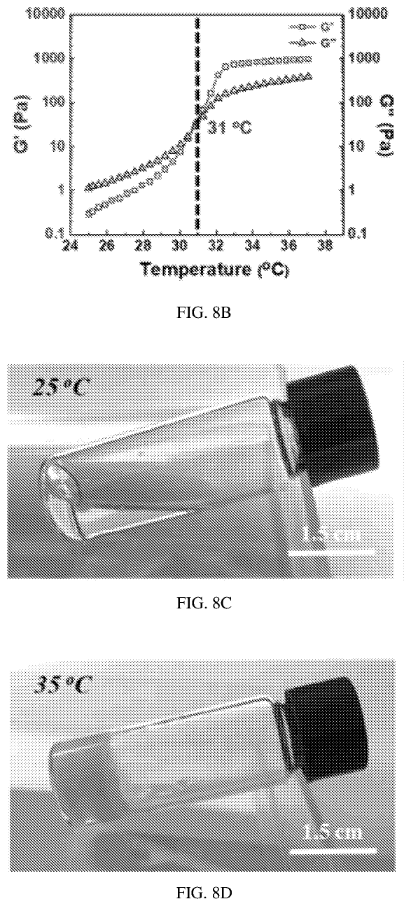

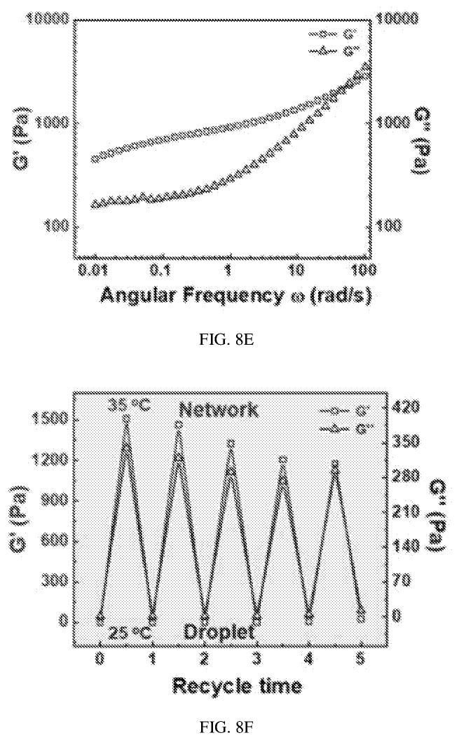

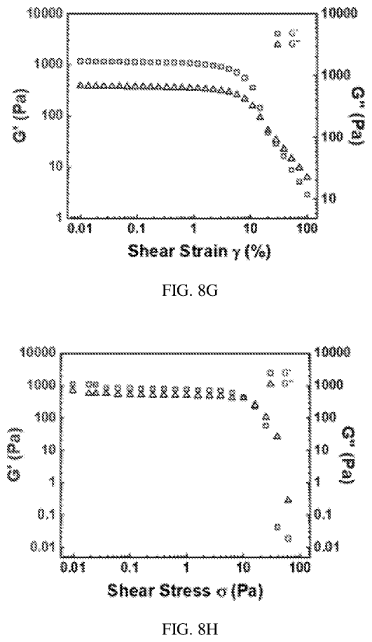

Rheological properties of the PNIPAM/DEX systems were systematically measured to quantify physio-chemical aspects of the transformation. The viscosity of 5 wt % PNIPAM aqueous solution and 5 wt % DEX aqueous solution are 165 mPa·s and 2.3 mPa·s, respectively, as illustrated in FIG. 8A. This 71-fold difference in viscosities indicates that the movement of PNIPAM chains is much slower than dextran chains due to the significant size disparity. An ATPS composed of 5 wt % PNIPAM and 5 wt % DEX was prepared and equilibrated. The viscosity of the PNIPAM-rich phase (33 mPa's) is still 7-fold higher than the DEX-rich phase (4.6 mPa·s). The significant difference in viscosities induced by large size disparity demonstrates a dynamic asymmetry between PNIPAM and DEX. The elastic modulus (G′) and viscous modulus (G″) of the PNIPAM-rich phase over the temperature range from 25° C. to 37° C. to describe its phase transition quantitatively, and plotted in FIG. 8B. The elastic modulus of the PNIPAM-rich phase under 25° C. is smaller than the viscous modulus, indicating the solution is liquid-like, as shown in FIG. 8C. As the temperature increases, the elastic modulus increases much faster than the viscous modulus. The two moduli intersect at approximately 31° C. When the temperature exceeds 31° C., the elastic modulus becomes larger than the viscous modulus, indicating the PNIPAM-rich phase is more solid-like and a network is generated, as shown in FIG. 8D. The elastic modulus and viscous modulus of the PNIPAM networks are 921 Pa and 291 Pa at 1 rad/s, respectively, as illustrated in FIG. 8E. The two moduli of the PNIPAM-rich phase were tested at 25° C. and 35° C. for five cycles, as shown in FIG. 8F. The G′ and G″ of PNIPAM-rich phase at 25° C. are only 0.1 Pa and 0.7 Pa at 10 rad/s, displaying a liquid-like state. By raising the temperature to 35° C., the G′ and G″ at 10 rad/s increase sharply to 1509 Pa and 340 Pa, respectively. A larger G′ than G″ indicates the formation of solid network. As the temperature decreases back to 25° C., the G′ and G″ at 10 rad/s dropped to 0.3 Pa and 0.9 Pa. A lower G′ than G″ implies that the PNIPAM networks are dissolved and turn to the original liquid-like state. The stability in variation tendency of G′ and G″ of PNIPAM-rich phase for five cycles demonstrates the reversibility of its phase transition between liquid droplets and solid networks.

The mechanics of the viscoelastic networks are characterized by measuring the variation of their elastic modulus under various stresses and strains. As shown in FIG. 8G and FIG. 8H, the viscoelastic network formed in the ATPS is both strain-softening and stress-softening. When the applied strain is over 10%, the elastic modulus of the viscoelastic network decreases sharply from 357 Pa at 10% of strain to only 3 Pa at 100% of strain (FIG. 8G). When the applied stress increases from 10 to 61 Pa, the elastic modulus of the viscoelastic network also displays a rapid decline from 437 Pa to 0.02 Pa, as demonstrated in FIG. 8H. The viscoelastic network displays similar stress weakening behavior as pure microtubule networks (see “Y.-C. Lin, G. H. Koenderink, F. C. Mackintosh, D. A. Weitz, Macromolecules 2007, 40, 7714.”) and weakly cross-linked actin networks (see “M. L. Gardel, K. E. Kasza, C. P. Brangwynne, J. Liu, D. A. Weitz, Methods Cell Biol. 2008, 89, 487.”).

Besides, adopting stimuli-responsive polymers as one component of ATPSs universally generate hydrophobic networks by applying this mechanism. Multiple thermal-responsive ATPSs by mixing PNIPAM with polyvinyl alcohol (PVA), polyethyleneimine (PEI), polyacrylamide (PAM), and ficoll (type 400). All these ATPSs can generate hydrophobic networks. Varying the concentration and temperature can further regulate properties of the hydrophobic networks, such as pore size, density, and viscoelasticity, which would broaden their potential applications.

### Interactions Between Dynamic Hydrophobic Networks and FUS Condensates

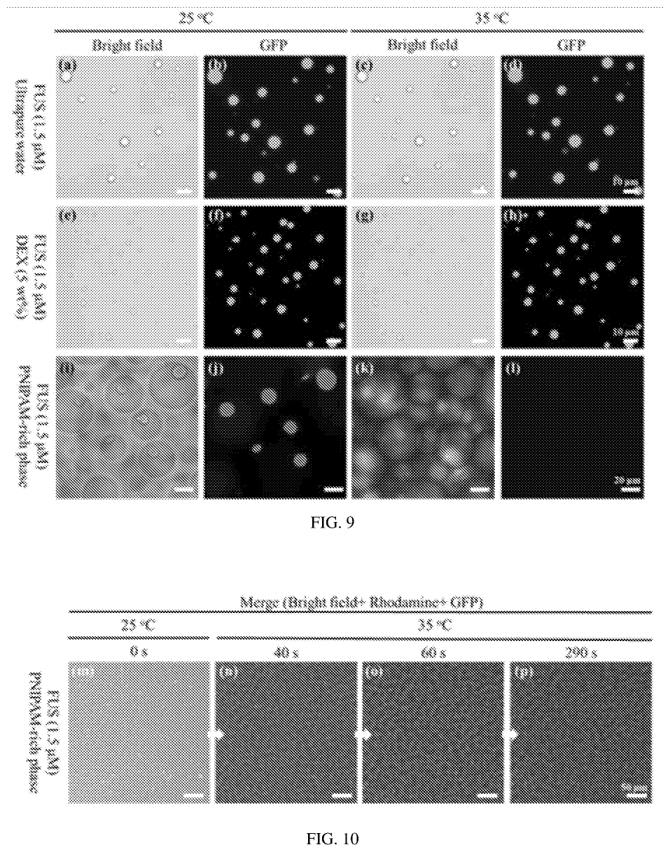

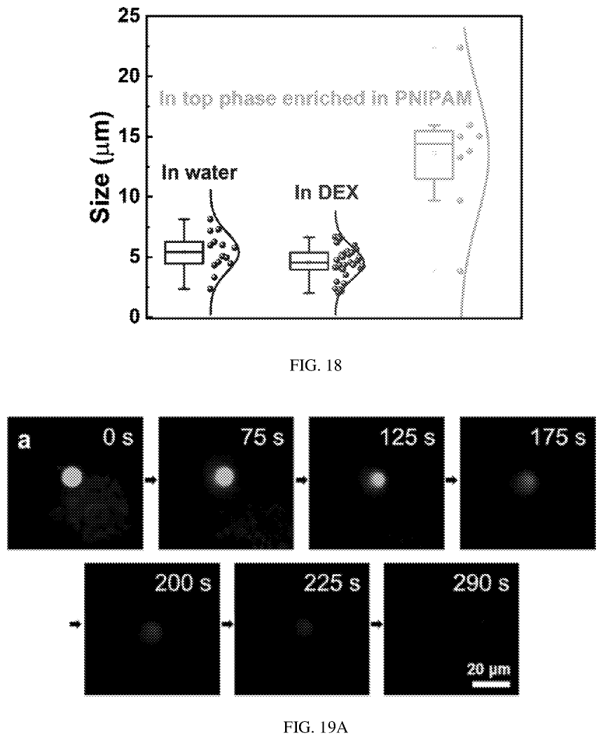

The PNIPAM networks formed from the aqueous liquid-liquid phase separation and liquid-solid phase separation (AqLL-LS PS²) of the new aqueous two-phase system can melt phase-separated FUS condensates effectively. By mixing 1 μL FUS proteins (30 μM) with 19 μL ultrapure water at 25° C., FUS condensates with diameters of 2.3-9.7 μm are generated, as shown in FIG. 18 and FIG. 9 blocks (a) and (b). The liquid-liquid phase separation (LLPS) of FUS proteins is driven by the reinforced multivalent interactions between intrinsically disordered regions due to the dilution of salt, which reduces electrostatic screening. The formed FUS condensates maintain their stability at 35° C., as shown in FIG. 9 blocks (c) and (d), indicating that heat does not lead to the disassembly of FUS condensates. When 1 μL FUS protein (30 μM) is mixed with 19 μL 5 wt % DEX aqueous solution, FUS condensates with diameters of 2.0-6.7 μm are separated from DEX solution. The increase in temperature of the DEX bulk phase from 25° C., as shown in FIG. 9 blocks (e) and (f), to 35° C., as shown in FIG. 9 blocks (g) and (h) does not change phase-separated FUS condensates, indicating that the stability of FUS condensates is independent of DEX. When 1 μL FUS protein (30 μM) is mixed with 19 μL PNIPAM-rich phase at 25° C., FUS condensates with diameters ranging from 9.7-22.4 μm are observed in phase-separated DEX droplets, as shown in FIG. 18 and FIG. 9 blocks (i) and (j). The enrichment of FUS protein in DEX droplets increases its local concentration, higher than 1.5 μM, while the concentration of the salt is the same under these three different situations. The partition of FUS protein into phase-separated DEX droplets increases its concentration, forming larger condensates. Therefore, FUS condensates in the PNIPAM-rich phase are larger than those in ultrapure water and DEX solution. As the temperature increases to 35° C., the hydrophobic PNIPAM networks form and dissolve FUS condensates, as shown in FIG. 9 blocks (k) and (1). The dissolution dynamics of FUS condensate is displayed in FIGS. 19A and 19B. The initial diameter of the FUS condensate is around 11 μm. When the temperature is increased to 35° C. to trigger the formation of hydrophobic PNIPAM networks, the FUS condensate starts to dissolve from the edge at 75 s. After increasing the temperature to 35° C. for 125 s, the size of FUS condensate decreases to 9 μm. When heated for 200 s, the FUS condensate further dissolves to 6.8 μm in diameter with significantly reduced fluorescence intensity. The FUS condensate is completely dissolved after 290 s. To visualize the interaction between FUS condensates and hydrophobic PNIPAM networks, the GFP signal of FUS and the rhodamine signal of PNIPAM are recorded simultaneously by a confocal microscope. A confocal microscope detects the fluorescent signals of PNIPAM and FUS protein simultaneously to visualize their dynamic interactions. After increasing the temperature to 35° C. for 40-60 s, the hydrophobic PNIPAM networks are generated and contact the FUS condensates, as shown in FIG. 10 blocks (m) through (o). The FUS condensates indicated by the green fluorescent protein (GFP) signal completely disappear after heating at 35° C. for 290 s. The hydrophobic networks interrupt the hydrophobic interaction between FUS molecules and thus lead to the dissolution of the condensates. The melting of the condensate may also result from viscoelastic constraints from the PNIPAM networks. Though truncating specific sequences among intrinsically disordered regions of FUS can also relieve the liquid-to-solid phase transitions, it fails to retain FUS normal function. However, by forming PNIPAM networks from the ATPS, phase-separated FUS condensates have been dissolved effectively without truncating any sequence. Hence, the ATPS system, according to an embodiment, can dissolve pathologically relevant biomolecular condensates formed by hydrophobic interactions.

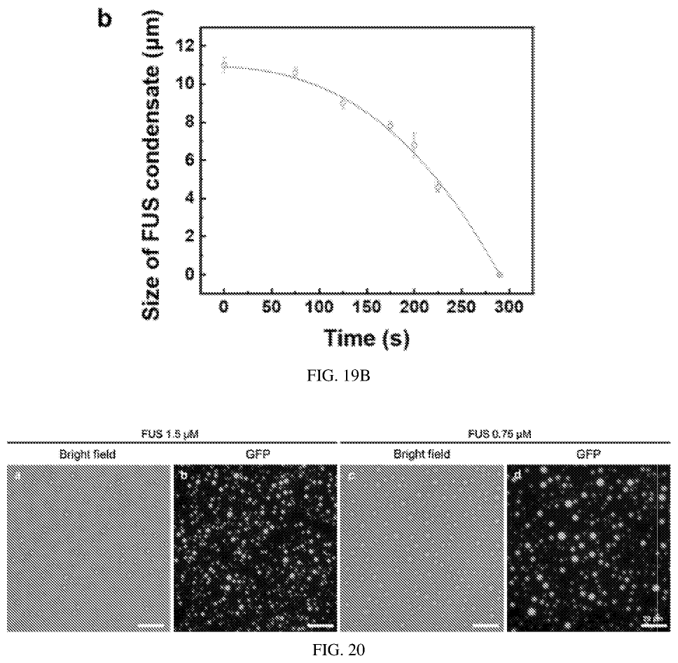

To test whether the dissolution of liquid FUS condensates is a result of dilution, dilution experiments are performed: 1 μL FUS stock solution is mixed with 19 μL ultrapure water homogeneously, and then injected into one chamber. Multiple phase-separated FUS condensates can be observed, as shown in subfigure a-b of FIG. 20. Then 20 μL ultrapure water is injected into the chamber to dilute the FUS mixture prepared previously. FUS condensates remains stable and even increase in size (subfigure c-d of FIG. 20). The results indicate that the disassembly of the liquid FUS condensates is not induced by the dilution. As PNIPAM chains reconfigure from hydrophilic coils to hydrophobic globules when the temperature exceeds its LCST. The reinforced polymeric interactions, such as hydrophobic interactions and hydrogen bond, between FUS protein and PNIPAM networks may dominate the dissolution of the liquid FUS condensates.

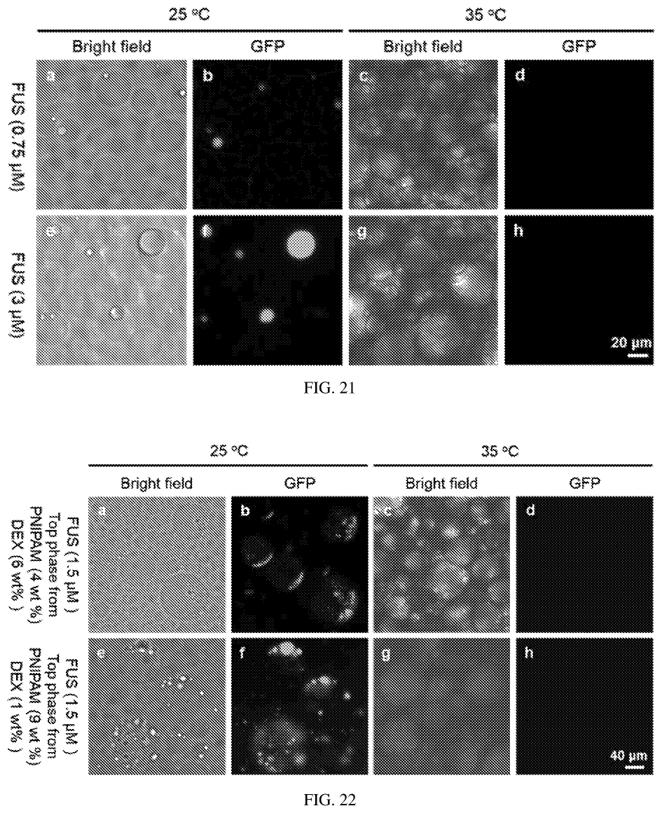

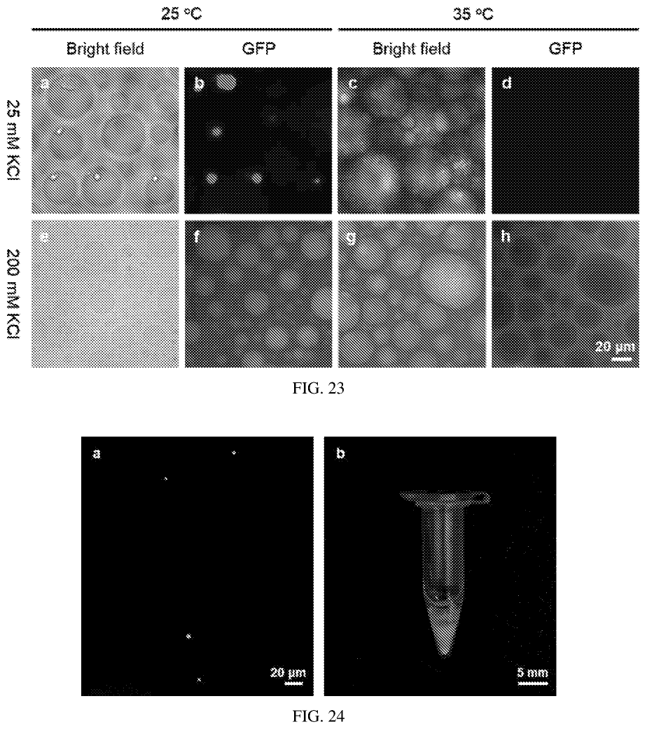

To comprehensively demonstrate the capability of the PNIPAM/DEX system to regulate the self-assembly of FUS condensates, the LLPS of FUS proteins is investigated under different protein concentrations, PNIPAM conditions, and salt concentrations. The results are in agreement with our proposed mechanism (FIG. 21, 22, and subfigures a-d of FIG. 23). It's worth noting that when the salt concentration is increased to 200 mM, FUS protein partitions into DEX droplets without condensation (subfigures e-f of FIG. 23). The strong electrostatic screening from salt restrains the LLPS of FUS protein. The result agrees well with the previous report. Raising the temperature to 35° C., the PNIPAM networks are formed (subfigure g of FIG. 23), accompanied by the adhesive of FUS protein on the networks, as confirmed by subfigure h of FIG. 23. This phenomenon corroborates that PNIPAM networks have a stronger interaction with FUS protein at 35° C. When the salt concentration is increased to 3 M, FUS protein separates and forms condensates (subfigure a of FIG. 24). Hydrophobic interactions dominate the phase separation of FUS protein at high salt concentrations. However, high salt concentration induces the precipitation of polymers (subfigure b of FIG. 24). The PNIPAM/DEX system cannot work when the concentration of salt is high than 0.3 M, probably due to the salting out effect. Though truncating specific sequences among intrinsically disordered regions of FUS can also relieve the liquid-to-solid phase transitions, it fails to retain FUS's normal function. However, by forming PNIPAM networks from the newly proposed ATPS, the phase-separated FUS condensates are dissolved without truncating any sequence. The understanding based on our system may have the potential to modulate the phase separation of biomolecules on-demand via external stimuli, which deserves further investigations.

### Thermally Reversible Dewetting/Wetting of an ATPS Droplet in Oil

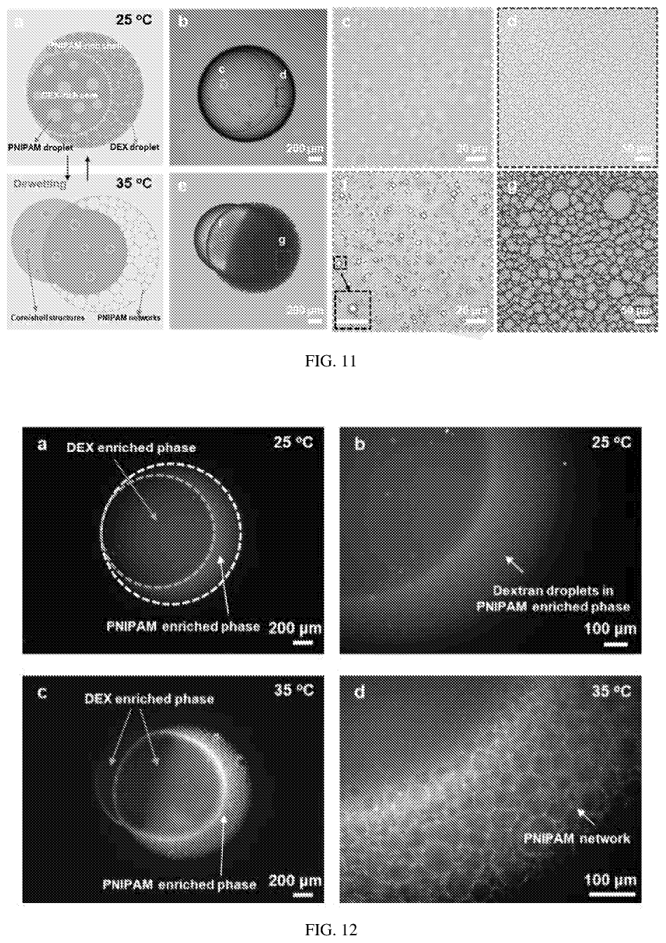

The PNIPAM/DEX droplet displays thermoreversible dewetting-wetting transition in oil. We demonstrate this by injecting a 2 μL heterogeneous ATPS (5 wt % PNIPAM and 5 wt % DEX) droplet into 3 mL hexadecane solution at room temperature. The bulk hexadecane with a smaller density covers the deposited ATPS droplet and avoids water evaporation. After 24 h, the PNIPAM/DEX droplet turns into a core/shell structure (subfigures a, b of FIG. 11). The ATPS droplets display a fluorescent shell enriched in PNIPAM labeled with rhodamine B and a dark core enriched in DEX, as shown in subfigure a of FIG. 12. Smaller PNIPAM and DEX droplets can be observed in the core and shell, respectively, as confirmed by the bright-field images in subfigures c, d of FIG. 11, as well as the fluorescence images in subfigure b of FIG. 12. The ATPS droplet dewets (subfigure e of FIG. 11) with a protrusion (dark part in subfigure c of FIG. 12) when the temperature is increased to 35° C. The reconfiguration of PNIPAM chains raises the surface energy between the DEX enriched core and the PNIPAM enriched shell, higher than that between the DEX enriched core and the external hexadecane phase, thereby inducing the dewetting. Core/shell (subfigure f of FIG. 11) and network structures (subfigure g of FIG. 11, subfigure d of FIG. 12) are formed in the DEX enriched core and the PNIPAM enriched shell, respectively. The dewetting process is reversible since PNIPAM chains can switch between hydrophilic coils and hydrophobic globules in response to temperature changes. By decreasing the temperature to 25° C. again, the hydrophobic PNIPAM networks dissolve, and the protruding phase retracts. The dewetting droplet recovers to its original core-shell structure. The PNIPAM/DEX system displays rich AqLL-LS PS² behaviors by forming diverse and complex hierarchical structures, such as liquid droplets and viscoelastic networks. The capability of the single system to mimic both liquid organelles and biological networks creates an ideal platform to investigate their dynamic interactions.

This application introduces an approach to generate long-live viscoelastic PNIPAM networks through aqueous liquid-liquid phase separation and liquid-solid phase separation (AqLL-LS PS²) of an aqueous two-phase system composed of thermo-responsive PNIPAM and DEX. The PNIPAM/DEX system displays ultrasensitive phase separation behaviors in response to variation in temperature. The transition temperature is around 31° C., which is close to the human body temperature and suitable for investigating aqueous phase separation involving biomolecules. Diverse morphologies, including spherical aggregates, hydrophobic networks, porous spheres, and core/shell structures, can be prepared by adjusting the composition and temperature. Besides, the formation of hydrophobic PNIPAM networks is thermally reversible since PNIPAM chains can switch between hydrophilic coils and hydrophobic globules by modulating the temperature. Furthermore, the formed hydrophobic PNIPAM networks dissolve liquid FUS condensates. This result agrees well with previous studies, in which 1,6-hexanediol, a frequently used hydrophobic disruptor, dissolves FUS condensates, our hydrophobic PNIPAM networks are able to dissolve the FUS condensates within 290 s. Compared with previously reported ATPSs, selecting stimuli-responsive polymers as one component of the ATPSs imparts a wider range of complex morphological changes. This is attributed to the reconfigurability of stimuli-responsive polymers in response to external stimuli. By selecting stimuli-responsive materials with specific hydrophilic-to-hydrophobic transitions, the hierarchical structures can be precisely triggered by different physico-chemical signals on-demand. For instance, adding photothermal agents, such as gold nanorods, graphene, MXene, etc., into ATPSs containing thermo-responsive materials will endow the system with photo-sensitive properties. The desired subcellular structures can be regulated in a contactless and spatiotemporal manner. Our intelligent ATPS provides a new model to construct viscoelastic networks that can be dynamically assembled and dissociated with the potential to mediate the phase separation of biomolecules. We believe there is more to be understood and inspired in regulating other liquid organelles by synthetic viscoelastic networks prepared from this system. The proposed ATPS also sheds light on developing advanced materials with cell-mimetic hierarchical structures that have the potential to regulate the liquid-liquid phase separation of biomolecules and thus control the self-assembly of biomolecular condensates on-demand.

All patents, patent applications, provisional applications, and publications cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

Martin, S. & Parton, R. G. Lipid droplets: a unified view of a dynamic organelle. Nature Reviews Molecular Cell Biology 7, 373-378 (2006).
Fletcher, D. A. & Mullins, R. D. Cell mechanics and the cytoskeleton. Nature 463, 485-492 (2010).
Ursell, T. S. et al. Rod-like bacterial shape is maintained by feedback between cell curvature and cytoskeletal localization. Proceedings of the National Academy of Sciences 111, E1025 (2014).
Sydiskis Robert, J. & Roizman, B. Polysomes and Protein Synthesis in Cells Infected with a DNA Virus. Science 153, 76-78 (1966).
Lyon, A. S., Peeples, W. B. & Rosen, M. K. A framework for understanding the functions of biomolecular condensates across scales. Nature Reviews Molecular Cell Biology 22, 215-235 (2021).
Huang, W. Y. C. et al. A molecular assembly phase transition and kinetic proofreading modulate Ras activation by SOS. Science 363, 1098 (2019).
Lafontaine, D. L. J., Riback, J. A., Bascetin, R. & Brangwynne, C. P. The nucleolus as a multiphase liquid condensate. Nature Reviews Molecular Cell Biology 22, 165-182 (2021).
Shan, Z. et al. Basal condensation of Numb and Pon complex via phase transition during Drosophila neuroblast asymmetric division. Nature Communications 9, 737 (2018).
Choquet, D., Felsenfeld, D. P. & Sheetz, M. P. Extracellular Matrix Rigidity Causes Strengthening of Integrin-Cytoskeleton Linkages. Cell 88, 39-48 (1997).
Hirokawa, N., Noda, Y., Tanaka, Y. & Niwa, S. Kinesin superfamily motor proteins and intracellular transport. Nature Reviews Molecular Cell Biology 10, 682-696 (2009).
Feric, M. & Brangwynne, C. P. A nuclear F-actin scaffold stabilizes ribonucleoprotein droplets against gravity in large cells. Nature Cell Biology 15, 1253-1259 (2013).
Banani, S. F., Lee, H. O., Hyman, A. A. & Rosen, M. K. Biomolecular condensates: organizers of cellular biochemistry. Nature Reviews Molecular Cell Biology 18, 285-298 (2017).
Alberti, S., Gladfelter, A. & Mittag, T. Considerations and Challenges in Studying Liquid-Liquid Phase Separation and Biomolecular Condensates. Cell 176, 419-434 (2019).
Alberti, S. & Hyman, A. A. Biomolecular condensates at the nexus of cellular stress, protein aggregation disease and ageing. Nature Reviews Molecular Cell Biology 22, 196-213 (2021).
Keating, C. D. Aqueous Phase Separation as a Possible Route to Compartmentalization of Biological Molecules. Accounts of Chemical Research 45, 2114-2124 (2012).
Sokolova, E. et al. Enhanced transcription rates in membrane-free protocells formed by coacervation of cell lysate. Proceedings of the National Academy of Sciences 110, 11692 (2013).
Andes-Koback, M. & Keating, C. D. Complete Budding and Asymmetric Division of Primitive Model Cells To Produce Daughter Vesicles with Different Interior and Membrane Compositions. Journal of the American Chemical Society 133, 9545-9555 (2011).
Dewey, D. C., Strulson, C. A., Cacace, D. N., Bevilacqua, P. C. & Keating, C. D. Bioreactor droplets from liposome-stabilized all-aqueous emulsions. Nature Communications 5, 4670 (2014).
Sah, H. Stabilization of proteins against methylene chloride/water interface-induced denaturation and aggregation. Journal of Controlled Release 58, 143-151 (1999).
Song, Y. et al. Budding-like division of all-aqueous emulsion droplets modulated by networks of protein nanofibrils. Nature Communications 9, 2110 (2018).
Song, Y. et al. Fabrication of fibrillosomes from droplets stabilized by protein nanofibrils at all-aqueous interfaces. Nature Communications 7, 12934 (2016).
Chao, Y. & Shum, H. C. Emerging aqueous two-phase systems: from fundamentals of interfaces to biomedical applications. Chemical Society Reviews 49, 114-142 (2020).
Zhu, S. et al. Rapid Multilevel Compartmentalization of Stable All-Aqueous Blastosomes by Interfacial Aqueous-Phase Separation. ACS Nano 14, 11215-11224 (2020).
Chao, Y., Mak, S. Y., Rahman, S., Zhu, S. & Shum, H. C. Generation of High-Order All-Aqueous Emulsion Drops by Osmosis-Driven Phase Separation. Small 14, 1802107 (2018).
Strulson, C. A., Molden, R. C., Keating, C. D. & Bevilacqua, P. C. RNA catalysis through compartmentalization. Nature Chemistry 4, 941-946 (2012).
Xie, G. et al. Compartmentalized, All-Aqueous Flow-Through-Coordinated Reaction Systems. Chem 5, 2678-2690 (2019).
Guo, W. et al. Non-associative phase separation in an evaporating droplet as a model for prebiotic compartmentalization. Nature Communications 12, 3194 (2021).
Ma, Q. et al. Cell-Inspired All-Aqueous Microfluidics: From Intracellular Liquid-Liquid Phase Separation toward Advanced Biomaterials. Advanced Science 7, 1903359 (2020).
Koppers, M., Özkan, N. & Farís, G. G. Complex Interactions Between Membrane-Bound Organelles, Biomolecular Condensates and the Cytoskeleton. Frontiers in Cell and Developmental Biology 8, 1661 (2020).
Shen, Y. et al. Effects of Vimentin Intermediate Filaments on the Structure and Dynamics of In Vitro Multicomponent Interpenetrating Cytoskeletal Networks. Physical Review Letters 127, 108101 (2021).
Zhang, T., Sanguramath, R. A., Israel, S. & Silverstein, M. S. Emulsion Templating: Porous Polymers and Beyond. Macromolecules 52, 5445-5479 (2019).
Stuart, M. A. C. et al. Emerging applications of stimuli-responsive polymer materials. Nature Materials 9, 101-113 (2010).
Chen, Y. et al. Resolving Optical and Catalytic Activities in Thermoresponsive Nanoparticles by Permanent Ligation with Temperature-Sensitive Polymers. Angewandte Chemie International Edition 58, 11910-11917 (2019).
Zhang, P. et al. A Microstructured Graphene/Poly (N-isopropylacrylamide) Membrane for Intelligent Solar Water Evaporation. Angewandte Chemie International Edition 57, 16343-16347 (2018).
Fernández-Castaño Romera, M. et al. Mimicking Active Biopolymer Networks with a Synthetic Hydrogel. Journal of the American Chemical Society 141, 1989-1997 (2019).
Sai, H. et al. Hierarchical porous polymer scaffolds from block copolymers. Science 341, 530-534 (2013).
Wang, F. et al. Progress Report on Phase Separation in Polymer Solutions. Advanced Materials 31, 1806733 (2019).
Cabral, J. T. & Higgins, J. S. Spinodal nanostructures in polymer blends: On the validity of the Cahn-Hilliard length scale prediction. Progress in Polymer Science 81, 1-21 (2018).
Li, L., Shen, X., Hong, S. W., Hayward, R. C. & Russell, T. P. Fabrication of Co-continuous Nanostructured and Porous Polymer Membranes: Spinodal Decomposition of Homopolymer and Random Copolymer Blends. Angewandte Chemie International Edition 51, 4089-4094 (2012).
Xiong, H. et al. A Polymer-Assisted Spinodal Decomposition Strategy toward Interconnected Porous Sodium Super Ionic Conductor-Structured Polyanion-Type Materials and Their Application as a High-Power Sodium-Ion Battery Cathode. Advanced Science 8, 2004943 (2021).
Meyer, E. E., Rosenberg, K. J. & Israelachvili, J. Recent progress in understanding hydrophobic interactions. Proceedings of the National Academy of Sciences 103, 15739 (2006).
Patel, A. et al. A Liquid-to-Solid Phase Transition of the ALS Protein FUS Accelerated by Disease Mutation. Cell 162, 1066-1077 (2015).
Krainer, G. et al. Reentrant liquid condensate phase of proteins is stabilized by hydrophobic and non-ionic interactions. Nature Communications 12, 1085 (2021).
Rosowski, K. A. et al. Elastic ripening and inhibition of liquid-liquid phase separation. Nature Physics 16, 422-425 (2020).
Wei, X., Zhou, J., Wang, Y. & Meng, F. Modeling Elastically Mediated Liquid-Liquid Phase Separation. Physical Review Letters 125, 268001 (2020).
Lin, Y. et al. Toxic PR Poly-Dipeptides Encoded by the C9orf72 Repeat Expansion Target LC Domain Polymers. Cell 167, 789-802.e712 (2016).
Lutz, J.-F., Akdemir, Ö. & Hoth, A. Point by Point Comparison of Two Thermosensitive Polymers Exhibiting a Similar LCST: Is the Age of Poly (NIPAM) Over? Journal of the American Chemical Society 128, 13046-13047 (2006).
Jain, K., Vedarajan, R., Watanabe, M., Ishikiriyama, M. & Matsumi, N. Tunable LCST behavior of poly(N-isopropylacrylamide/ionic liquid) copolymers. Polymer Chemistry 6, 6819-6825 (2015).
Du, X. et al. Photothermally Triggered Shape-Adaptable 3D Flexible Electronics. Advanced Materials Technologies 2, 1700120 (2017).
Du, X. et al. Reconfiguration, Camouflage, and Color-Shifting for Bioinspired Adaptive Hydrogel-Based Millirobots. Advanced Functional Materials 30, 1909202 (2020).
Hu, Y. et al. Hydrophilicity-Hydrophobicity Transformation, Thermoresponsive Morphomechanics, and Crack Multifurcation Revealed by AIEgens in Mechanically Strong Hydrogels. Advanced Materials 33, 2101500 (2021).
Keerl, M., Smirnovas, V., Winter, R. & Richtering, W. Interplay between Hydrogen Bonding and Macromolecular Architecture Leading to Unusual Phase Behavior in Thermosensitive Microgels. Angewandte Chemie International Edition 47, 338-341 (2008).
Tamai, Y., Tanaka, H. & Nakanishi, K. Molecular Dynamics Study of Polymer-Water Interaction in Hydrogels. 1. Hydrogen-Bond Structure. Macromolecules 29, 6750-6760 (1996).
Hou, L. & Wu, P. LCST transition of PNIPAM-b-PVCL in water: cooperative aggregation of two distinct thermally responsive segments. Soft Matter 10, 3578-3586 (2014).
Lin, D. et al. Sol-gel transition of polyamic acid solution during chemical imidization and its effect on the drawing behavior of polyimide films. Polymer 226, 123842 (2021).
Style, R. W. et al. Liquid-Liquid Phase Separation in an Elastic Network. Physical Review X8, 011028 (2018).
Mace, C. R. et al. Aqueous Multiphase Systems of Polymers and Surfactants Provide Self-Assembling Step-Gradients in Density. Journal of the American Chemical Society 134, 9094-9097 (2012).
Shen, Y. et al. Biomolecular condensates undergo a generic shear-mediated liquid-to-solid transition. Nature Nanotechnology 15, 841-847 (2020).
Wang, C., Flynn, N. T. & Langer, R. Controlled Structure and Properties of Thermoresponsive Nanoparticle-Hydrogel Composites. Advanced Materials 16, 1074-1079 (2004).

1. A biological network mimic, comprising an aqueous two-phase system (ATPS) comprising stimulus-responsive polymer and a non-responsive polymer, wherein multiple phase-separated structures are generated under external stimuli.

2. The biological network mimic according to claim 1, wherein the generation of phase-separated structures is reversible.

3. The biological network mimic according to claim 1, wherein the stimulus is a temperature change, a pH change, an irradiation with light, application of an electric field, or application of a magnetic field.

4. The biological network mimic according to claim 1, further comprising a photothermal agent.

5. The biological network mimic according to claim 4, wherein the photothermal agent comprises gold nanorods (GNRs), graphene, MXene, and/or carbon nanotubes.

6. The biological network mimic according to claim 1, wherein the stimulus-responsive polymer is poly(N-isopropylacrylamide) (PNIPAM) and the non-responsive polymer is dextran (DEX).

7. The biological network mimic according to claim 6, wherein the stimulus is a temperature change.

8. The biological network mimic according to claim 1, wherein the stimulus-responsive polymer is an Elastin-like polypeptide or poly(N-vinylcaprolactam) for the stimulus of a temperature change.

9. The biological network mimic according to claim 1, wherein the stimulus-responsive polymer is poly(acrylic acid) or hyaluronic acid for the stimulus of a pH change.

10. The biological network mimic according to claim 1, wherein the stimulus-responsive polymer is a spiropyran-, azobenzene-, or dithienylethene-derived polymer for the stimulus of an irradiation with light.

11. The biological network mimic according to claim 1, wherein the generation of the multiple phase-separated structures is an ultrasensitive thermo-induced aqueous liquid-liquid phase separation or a liquid-solid phase separation (AqLL-LS PS²).

12. The biological network mimic according to claim 1, wherein the multiple phase-separated structure comprises solid spheres, porous networks, hollow spheres, or core-shell spheres.

13. A method for probing interactions between dynamic networks and biomolecular condensates in vitro, comprising:
providing a biological network mimic according to claim 1;
combining the biological network mimic with at least one biomolecule; and
stimulating the biological network mimic.

14. The method according to claim 13, wherein the biological network mimic is a combination of poly(N-isopropylacrylamide) (PNIPAM) and dextran (DEX).

15. The method according to claim 13, wherein the biomolecule is a FUS protein.
